(19) United States
(12) Patent Application Publication
(10) Pub. No.: US 2002/0151520 A1
(43) Pub. Date: Oct. 17, 2002

(54) Method of promoting angiogenesis

(76) Inventor: Elliott A. Gruskin, Killingworth, CT (US)

Correspondence Address: CARTER, DELUCA, FARRELL and SCHMIDT, LLP, 445 BROAD HOLLOW ROAD, SUITE 225, MELVILLE, NY, 11747, US

(21) Appl. No.: 09/533,833

(22) Filed: Mar. 23, 2000

Related U.S. Application Data
(60) Provisional application No. 60/126,166, filed on Mar. 25, 1999.

(51) Int. Cl. C07H 1/00; A61K 31/715; A01N 43/04; A61K 31/727; C08B 37/02

(52) U.S. Cl. 514/53; 514/54; 514/56; 514/61; 536/1.11; 536/112

(57) ABSTRACT

Methods of promoting angiogenesis are provided which include administering to a suitable locus a cross-linked polysaccharide having a positive charge. Preferred cross-linked polysaccharides are biodegradable beads. A positive charge may be provided by diethylaminoethyl (DEAE) groups associated with the polysaccharide.

### BACKGROUND

1. Technical Field

This disclosure relates to a method of promoting angiogenesis. More particularly, the disclosure relates to promoting angiogenesis using cross-linked polysaccharides having a positive charge.

2. Background of Related Art

Angiogenesis refers to the formation of new blood vessels. It plays a critical role in various biological processes such as wound healing, embryological development, the menstrual cycle, and inflammation and the pathogenesis of various diseases such as cancer, diabetic retinopathy, and rheumatoid arthritis, as described, e.g., in Folkman et al., Science 235: 442-447, 1987.

Accordingly, manipulation of angiogenesis represents a therapeutic approach by which to treat or prevent various conditions or diseases involving angiogenesis. For example, promotion of angiogenesis can aid in accelerating various physiological processes and treatment of diseases requiring increased vascularization such as the healing of wounds, fractures, and burns, inflammatory diseases, ischemic heart and peripheral vascular diseases, and myocardial infarction. Inhibition of angiogenesis can aid in the treatment of diseases such as cancer, diabetic retinopathy, and rheumatoid arthritis, where increased vascularization contributes toward the progression of such diseases.

The angiogenic process is regulated by interactions between stimulators and inhibitors of angiogenesis and is initiated by various stimuli such as ischemia, hypoxia, inflammation, etc. as described, e.g., Melillo et al., Cardiovascular Research 35: 480-489, 1997, and Battegay, J. Molecular Medicine, 73:333-46, 1995. The process involves a sequence of events which includes protease secretion by endothelial cells, degradation of the basement membrane, migration of endothelial cells toward the tissue to be vascularized, proliferation and differentiation of endothelial cells, and synthesis of a new basement membrane as described, e.g., in Furcht et al., Lab. Invest., 55: 505-509, 1986; Liotta et al., Cell 64: 327-336, 1994; and Melillo et al., supra.

Several model systems for measuring angiogenesis in vivo have been developed. The chick chorioallantoric membrane assay (“CAM” assay) measures angiogenesis of a test substance in an embryonic system, whereas the rabbit corneal pocket assay measures angiogenesis in mature systems. Both assays have been described, e.g., in Catsimpoolas et al., U.S. Pat. No. 4,888,324, and Bentley, U.S. Pat. No. 5,356,874, both of which are incorporated herein by reference. Recently, another system of measuring in vivo angiogenesis uses a liquid matrix material known as MATRIGEL which forms a gel when injected into a host, as described, e.g., in Passaniti et al., U.S. Pat. No. 5,382,514, which is incorporated herein by reference.

A variety of peptide factors are known to promote angiogenesis such as acidic and basic fibroblast growth factor, alpha and beta tumor necrosis factor, platelet-derived growth factor, vascular endothelial cell growth factor, angiogenin and others as described, e.g., in Battegay, supra; and Folkman, Ann. NY Acad. Sci. 401; 212-227, 1982.

Other factors, which are not proteins have also been shown to promote angiogenesis. These include prostaglandins E1 and E2 as described, e.g., in Ben-Ezra et al., Am J. Opthamol. 86: 445-461, 1978; oligosaccharides as described, e.g., in McCluer et al., U.S. Pat. No. 4,895,838; lipid-containing molecules such as gangliosides as described, e.g., in Catsimpoolas et al., U.S. Pat. No. 4,888,324; and omega-3 polyunsaturated fatty acids as described, e.g., in Kamarei et al., U.S. Pat. No. 4,879,312.

While these factors have been effective in promoting angiogenesis, improved methodologies and different biologic approaches are desirable for treating or preventing various conditions and/or diseases involving angiogenesis.

The polysaccharide, cross-linked dextran, commercially known as DEBRISAN®, has been utilized in the treatment of wounds for removal of foreign bodies, pus, exudates and damaged tissue from wound areas as described, e.g., in Gruskin et al., U.S. Pat. No. 5,502,042, incorporated herein by reference. Biodegradable cross-linked polysaccharides having a chemically induced charge have also been utilized to treat wounds as described in Gruskin et al., supra; to stimulate formation of bone, as described, e.g., in Bao et al., U.S. Pat. No. 5,263,985 and Eppley et al., U.S. Pat. No. 4,988,358; and to stimulate soft connective tissue growth and repair as described, e.g., in Eppley et al., U.S. Pat. No. 5,092,883.

It has now been unexpectedly shown that cross-linked polysaccharides having a positive charge promote angiogenesis.

### SUMMARY

Methods of promoting angiogenesis are provided herein which are useful in treating and preventing various conditions and diseases involving angiogenesis.

A method of promoting angiogenesis is provided which includes administering an effective angiogenesis promoting amount of a cross-linked polysaccharide having a positive charge to a desired locus, sufficient to promote angiogenesis in the desired locus.

### BRIEF DESCRIPTION OF THE DRAWING

FIG. 1 is a bar graph representing [³H]-proline incorporation based on specified cross-linked polysaccharide beads which corresponds to angiogenic activity of the respective polysaccharide beads.

### DETAILED DESCRIPTION OF PREFERRED EMBODIMENTS

Methods of promoting angiogenesis are described herein which involve administering an effective angiogenesis promoting amount of a cross-linked pqlysaccharide having a positive charge to a desired locus, sufficient to promote angiogenesis at the desired locus. As stated above, angiogenesis is an integral component of various important biological processes, such as wound healing, tumor growth, embryonic development, and inflammation and contributes to the pathogenesis of various diseases. Accordingly, the present methods of promoting angiogenesis using cross-linked polysaccharides as described herein, provide effective techniques for treating and/or preventing various conditions and diseases involving angiogenesis.

In addition, the present method of promoting angiogenesis provides a tool to analyze particular diseases in which angiogenesis is not desired such as cancer and diabetic retinopathy.

As stated above, cross-linked polysaccharides having a chemically induced charge have been used to treat wounds as described in Gruskin et al., supra; to stimulate formation of bone, as described, e.g., in Bao et al., U.S. Pat. No. 5,263,985 and Eppley et al., U.S. Pat. No. 4,988,358; and to stimulate soft connective tissue growth and repair as described, e.g., in Eppley et al., U.S. Pat. No. 5,092,883. The art, however, contains no mention of cross-linked polysaccharides having a positive charge useful for promoting angiogenesis when administered in an effective angiogenesis promoting amount.

It is contemplated that the cross-linked polysaccharide can be in various forms, e.g., a particle, flake, or bead, having varying shapes, e.g., a substantially spherical bead. Preferably, the cross-linked polysaccharide having a positive charge is a bead. The polysaccharide can be ionically or covalently cross-linked. Suitable polysaccharides that can be ionically cross-linked are well-known in the art and include alginic acid and pectic acids which complex with particular multivalent ions such as Ca⁺⁺ to provide ionic crosslinking. Preferably, the polysaccharide is covalently cross-linked, and includes polysaccharides such as dextran and modified alginates. More preferably, the covalently cross-linked polysaccharide is cross-linked dextran, which is commercially available, e.g., under the tradename, SEPHADEX from Pharmacia Corp., (Piscataway, N.J.), which is a bead. Modified alginates may be prepared as described in PCT WO 93/09176, which is incorporated herein by reference.

Preferably the cross-linked polysaccharide is biodegradable. Techniques for preparing biodegradable cross-linked polysaccharides are well known in the art. For example, biodegradable cross-linked polysaccharide can be produced by oxidizing the cross-linked polysaccharide to produce linkages which are unstable under hydrolytic conditions as described, e.g., in Gruskin et al., supra incorporated herein by reference. When the cross-linked polysaccharide is rendered biodegradable by oxidation, preferably, the positive charge provided on the cross-linked polysaccharide is induced on the polysaccharide prior to oxidation.

A positive charge can be provided on the polysaccharide by reaction with suitable functional groups, e.g. diethylaminoethyl (DEAE) groups, using techniques that are well known in the art as described, e.g., in Eppley et al., U.S. Pat. No. 5,092,883 and Eppley et al., U.S. Pat. No. 4,988,358, both of which are incorporated herein by reference. Cross-linked dextran having DEAE groups is commercially available as a bead under the tradename DEAE-SEPHADEX from Pharmacia Corp., (Pistcataway, N.J.). More preferably, the cross-linked dextran having DEAE groups is biodegradable.

Examples of other cross-linked polysaccharides suitable for use herein that are available in bead form include Sepharose and Sephacel beads of Pharmacia Corp. (Piscataway, N.J). Both types of beads maybe provided with the DEAE functional group. The Sepharose beads are derived from agarose while the Sephacel beads are derived from cellulose.

The cross-linked polysaccharide beads may be porous. The porosity of cross-linked polysaccharide beads is dependent on the amount of cross-linking and the concentration of charged groups linked thereon. Any degree of porosity may be utilized in accordance with the methods described herein. In one embodiment, the average pore-size of such beads is about 500 Å. Commercially available cross-linked dextran beads which are employed in the compositions utilized herein have appropriate porosity, e.g., DEAE-SEPHADEX of A-, C- and G-25, -50.

While the present methods of promoting angiogenesis can be practiced in vitro, they are especially useful in in vivo applications. Accordingly, the present disclosure contemplates assays and/or treatment of various conditions or diseases involving angiogenesis which include, but are not limited to burns, wounds, fractures, cardiovascular disease, stroke, peripheral vascular disease, and alopecia.

With respect to in vivo use of the present methods, promotion of angiogenesis occurs when the cross-linked polysaccharide having a positive charge, alone or contained in a composition having a pharmaceutically acceptable carrier, is administered to an animal, e.g., a human, in an effective angiogenesis promoting amount. The amount will vary, based upon the size of the locus being treated, the severity of the wound or disease, and the animal's condition or disease, age, size and general physical condition. This dosage may be administered at one time, or in several discrete doses during the course of a day.

As stated above, the present method of promoting angiogenesis is also useful in in vitro applications. For example, the present method may be useful in analyzing specific mechanisms of angiogenesis in various endothelial cell cultures well known in the art, e.g., human umbilical vein endothelial cells (HUVEC).

The cross-linked polysaccharide having a positive charge can be administered in any suitable manner. One skilled in the art will appreciate that suitable methods of administering the cross-linked polysaccharide having a positive charge will depend on the extent and nature of the condition or disease to be treated.

The cross-linked polysaccharide having a positive charge may be administered directly to the site requiring promotion of angiogenesis, e.g., a diseased area or a wound or burn site. This means that the route of administration such as injections and topical administration are particularly suitable. For example, a surgeon can make an incision to expose a desired area and the polysaccharide is applied directly to the desired locus using a spatula, syringe, or by sprinkling. These techniques can be applied to cartilagenous areas, to ligamentous areas, organs, or any other internal locations in a patient where angiogenesis is desired. In addition, in the case of a surface wound, the polysaccharide can be applied directly to the locus of the wound site and optionally covered. It is thus also contemplated that the polysaccharide can be applied in the form of a dressing to the wound site.

The polysaccharide can be applied directly, without a vehicle or can be applied in the form of, e.g., a powder, gel, ointment, paste, fluid or lotion. Methods of incorporating substances into the aforementioned dosage forms are well known in the art and are described, e.g., in Remington's Pharmaceutical Sciences, 17th ed., Mack Publishing Company, 1985, incorporated herein by reference. One skilled in the art would recognize that various carriers, diluents, adjuvants, and so forth may be used in combination with the polysaccharides in order to enhance their delivery. All such combinations and variations are embraced by the present method of promoting angiogenesis.

When the cross-linked polysaccharide having a positive charge is mixed with a fluid to form a paste or fluid, any suitable pharmaceutically acceptable biocompatible fluid may be utilized. Appropriate vehicles should minimize irritation and inflammation at the site of administration. Thus, methods known to those skilled in the art such as providing isotonicity and non-irritating solvents such as polypropylene glycol are suitable. When the biocompatible fluid is water, saline or some other polar fluid, it may be necessary to take steps to avoid premature hydrolysis of biodegradable cross-linked polysaccharides, e.g., oxidized cross-linked positively charged polysaccharides as described in Gruskin et al., supra. For example, the composition can be provided as two separate components, namely the dry component (the biodegradable cross-linked polysaccharide having a positive charge) in one container and the fluid component in another container. The contents of the two containers are mixed shortly (preferably less than one hour) before application to the traumatized site. As another example, after mixing the biodegradable polysaccharide and a polar fluid, the composition can be frozen to avoid premature hydrolysis. The composition can be thawed shortly before application to a desired site.

Alternatively, the cross-linked polysaccharide can be mixed with a non-polar fluid. Suitable non-polar fluids include, mineral oil, non-ionic surfactants, liquid low molecular weight poly(ethylene oxide) and liquid low molecular weight poly(propylene oxide).

The cross-linked polysaccharide is preferably sterile and can be sterilized using any technique which does not expose the polysaccharide or composition containing the polysaccharide to excessively hydrolyzing conditions. Accordingly, ethylene oxide or gamma radiation are examples of preferred sterilization methods.

The present methods may also incorporate one or more medico-surgically useful substances or therapeutic agents, e.g., those which can further intensify the angiogenic response, and/or accelerate and/or beneficially modify the healing process when the composition is applied to the desired site requiring angiogenesis. For example, to further promote angiogenesis, repair and/or tissue growth, at least one of several hormones, growth factors or mitogenic proteins can be included in the composition, e.g., fibroblast growth factor, platelet derived growth factor, macrophage derived growth factor, etc. In addition, antimicrobial agents can be included in the compositions, e.g., antibiotics such as gentamicin sulfate, or erythromycin. Other medico-surgically useful agents can include anti-inflammatories, analgesics, anesthetics, rubifacients, enzymes, antihistamines and dyes.

The following examples are included for purposes of illustrating certain embodiments and are not intended to limit the scope of this disclosure.

The results from the following experiments demonstrate that a cross-linked polysaccharide having a positive charge, alone or contained in a composition having a pharmaceutically acceptable carrier, causes angiogenesis in a dose dependent manner when administered in an effective angiogenesis promoting amount.

The results were obtained using the chicken chorioallantoic membrane assay (“CAM” assay), and a quantitative method of evaluating angiogenesis in the CAM assay, i.e., [³H]-proline incorporation. In addition, sections of chorioallantoic membrane were stained with Haematoxylin and Eosin for histological examination.

### EXAMPLE 1

### CAM Assay

Different types of charged dextran beads: positive (SEPHADEX: DEAE, A-25 biodegradable and nonbiodegradable, and A-50), negative (SEPHADEX: CM, C-25 and C-50) and neutral (SEPHADEX G-25 and G-50) beads, were tested for angiogenic activity in the CAM assay, as described in West et al., Science 228: 1324-26, 1985, incorporated herein by reference. Each type of beads was suspended in sterile Dulbecco's A Phosphate-buffered saline (PBS), at a concentration of 50 mg/ml, and allowed to equilibrate overnight, at 4° C. This stock suspension was further diluted with sterile PBS, to give working suspensions of 25, 10 and 2 mg/ml, or 100, 40 and 8 μg/ pellet, respectively.

For application to the eggs, an aliquot of each sample was mixed with an equal volume of sterile 1% methylcellulose (Sigma M-0512, 4,000 centipoises). 8 μl of this suspension was then applied on to a 2 mm diameter “Teflon” column and dried, under sterile conditions, to give a clear disc. The samples were applied onto the CAM on day 10, when vessel growth has ostensibly finished and the angiogenic reaction determined on day 14. For histochemical examination, the membranes were fixed in situ, overnight at 4° C., using ice-cold 4% paraformaldehyde, in PBS, injected both under the membrane and on top of the membrane. Membranes were excised, embedded in paraffin, by standard techniques, and 5 μm sections were stained with Haematoxylin and Eosin.

The results from the CAM assay are summarized in Table 1. In general, DEAE SEPHADEX beads were more angiogenic than CM or G-25 SEPHADEX beads. In particular, the biodegradable DEAE SEPHADEX beads were highly angiogenic, over the range 100, 40 and 8 μg/ pellet (Table 1). Initial observations suggested that biodegradable DEAE-SEPHADEX A-25 was inflammatory in the CAM assay. However, closer examination indicated that the “pale yellow” color of the sample pellet was probably due to the absorption of a tissue exudate, similar in nature to serum. Non-biodegradable DEAE SEPHADEX A-25 beads showed weak to moderate activity, over the range 100, 40 and 8 μg/pellet, eliciting the growth of microvessels with very little effect on the larger vessels (Table 1). In contrast, CM- SEPHADEX C-25, SEPHADEX G-25 were essentially negative, or very weakly angiogenic, over the same range.

The larger pore-size C-50 and G-50 dextran beads seemed slightly more active than the smaller pore-size C-25 beads, but the smaller pore-size A-25 beads were more angiogenic than the larger pore size A-50 beads.

### EXAMPLE 2

### [³]-Proline Incorporation Assay

To obtain a quantitative assessment of angiogenesis, collagen synthesis was determined. Maragoudakis et al., Vascular Res. 50:215-22, 1995, have recently show that collagen synthesis correlates with vessel formation in the CAM assay. Collagen synthesis was determined by a modification of the method of Maragoudakis et al., J. Pharmacol. Exp. Therapeutics 251: 679-82, 1989, incorporated herein by reference. Essentially, samples were applied to the CAM, as outlined above, and 4 μl of aqueous [³H]-proline (1 μCi) was immediately applied on to the sample pellet. The angiogenic activity of each sample was assessed on day 13 and the membranes carefully excised. Using a sharp, sterile 1 cm diameter cork-borer, a circle of each membrane (1 cm diameter and approximately 5 mm radius from the point of sample application) was cut and transferred into 3 ml of ice-cold 5% trichloroacetic acid (TCA). After 4 hours at 4° C, the membranes were recovered by centrifugation at 10,000 g for 10 minutes, at 4° C. The membranes were washed twice with ice-cold 5% TCA, followed by three washes with 1 M NaCl in PBS. 1 ml of sterile collagenase solution (Sigma Type II, 1 mg/ml in Dulbecco's Modified Essential Medium, containing penicillin and streptomycin) was added and the digestion mixture incubated at 37° C. for 16 hours. The digestion was terminated, and undigested protein was precipitated, by the addition of 1 ml of ice-cold 20% TCA. After 4 hours at 4° C., the digest was centrifuged at 10,000 g for 20 minutes and an aliquot of the supernatant was counted in a liquid scintillation counter, to determine [³H]-proline incorporation into collagen peptides.

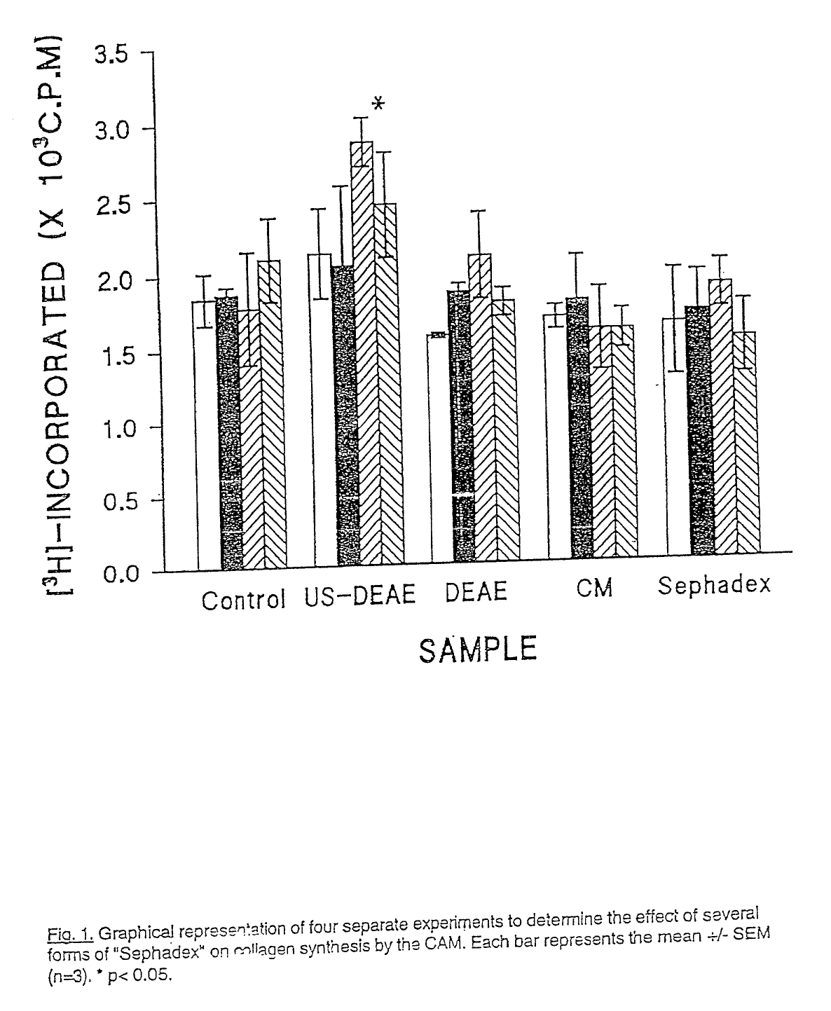

The results of [³H]-Proline incorporation for each type of charged bead are summarized in FIG. 1. [³H]-Proline incorporation was the same for the control, CM-SEPHADEX and SEPHADEX treated CAM. DEAE-SEPHADEX treated CAM generally showed a higher level of incorporation, but those treated with biodegradable DEAE-SEPHADEX showed a substantial increase over the control values. Although the increase was only significant in one experiment out of four, the trend does follow that of the semi-quantitative assessment of angiogenesis, i.e. control <SEPHADEX=CM-SEPHADEX<DEAE-SEPHADEX<biodegradable DEAE-SEPHADEX.

### EXAMPLE 3

### Histological Examination of the CAM Membranes

In several experiments, CAMs were fixed in situ, paraffin embedded, and transverse sections cut as close as possible to the site of sample application. These were then routinely stained with Haematoxylin and Eosin. The normal 14 day CAM is a thin membrane, with a distinct purple staining epithelial layer on the upper surface and a thinner epithelial layer at the inner surface. Between the epithelial layers is a thicker, reasonably cellular, connective tissue region containing discrete vessels, of various sizes.

In general, histological examination of the upper epithelial layer CAMs confirmed that the number of small vessels around the application sites reflected the overall level of angiogenesis. One possible exception appeared to be CM-SEPHADEX, which induced a very localized increase in microvessels just under the surface of the pellet, i.e., CM-SEPHADEX appeared to have a short range effect.

In particular, application of 40-100 μg of DEAE-SEPHADEX A-25 resulted in a general thickening of the membrane and an increase in both large and small vessels. Some of the vessels appeared not to be functional, i.e. they resembled vacuoles with no obvious cellular content, but similar structures were evident in the normal membrane. Vessels close to the sample beads appeared to be “leaky” with a definite extravasation of cells, possibly just red blood cells, but eosinophil-like cells and mast cells were commonly seen in electron-micrographs of the angiogenic CAM. At the application site, the beads apparently “sunk” into the membrane, but closer examination showed a pink/purple staining layer surrounding many of the beads. In most cases, this layer appeared cellular and a definite “skin” grew over the beads in some areas.

CM-SEPHADEX C-25 caused a more localized thickening of the CAM membrane and an increase in the number of small vessels at the site of application. This was much more discrete than that seen with DEAE, being more evident adjacent to the beads themselves. As with DEAE-beads, the membrane appeared to be growing around the beads, but in this case the growth appeared to be less epithelial directed and largely connective tissue. The neutral SEPHADEX G-25 beads appeared to have a similar, but less pronounced, effect as the CM-SEPHADEX beads. However, the growth around the beads seemed to involve the epithelial layer, rather than the matrix cells. The larger pore-size beads generally had less effect on the CAM, as exemplified by the DEAE-SEPHADEX A-50. Although there is a localized thickening of the membrane, there were few small vessels and only a limited epithelial “overgrowth”. The beads were larger and some mechanical disruption of the upper membrane appeared to have occurred, i.e. some beads appeared to be inside “broken” vacuoles or vessels.

The biodegradable DEAE-SEPHADEX A-25 beads caused very extensive changes in the membranes, far in excess of any encountered with the other types of bead. While a significant increase in the number of small vessels is evident, the major changes appeared to involve the epithelial layer and the connective tissue cells. Every membrane stained from this series showed a bright red staining associated with the upper epithelial layer. This may represent solubilized DEAE-SEPHADEX, as in some sections it is most dense close to the individual beads, but the presence of exudate (noted above) suggested that this was partly due to extravasated fibrinogen/fibrin deposits. The epithelial layer was greatly expanded by, what appeared to be, a localized epithelial cell proliferation. In most cases a substantial proliferation of connective tissue elements was seen and an amorphous unstained layer clearly separated the tissue undergoing remodeling from the underlying “uninvolved” tissue. There is little evidence that these changes are inflammatory, as few obvious inflammatory cells are present in the matrix, or around blood vessels.

To summarize the findings of the histological examination, the CM- and neutral SEPHADEX beads, of both pore-sizes, were least active, causing only a minor thickening, or proliferation. DEAE-SEPHADEX A-25 was more active than the large-pore A-50, inducing a definite thickening and overgrowth of the epithelial cells. With biodegradable DEAE-SEPHADEX A-25, a gross overgrowth of the epithelial layer was consistently present but, in addition, a stimulation of the underlying matrix formation and increased angiogenesis were also present. Without wishing to be bound by any particular theory, this difference in activity between the two DEAE-SEPHADEX preparations may be due to the release of soluble DEAE-dextran. It is unclear whether the matrix activation and angiogenesis are due to a direct stimulation by this soluble DEAE-dextran, or to factors released by the “hyperplastic” epithelium. Stimulation of a matrix “wound healing” reaction is accompanied by synthesis of matrix components such as collagen, evidenced by [³H]-proline incorporation, and amorphous matrix, probably hyaluronan. Inflammation does not seem to be a major component in this tissue reaction, as few obvious inflammatory cells were evident in the matrix, or near blood vessels.

TABLE 1
The angiogenic activity of charged Dextran beads

| | Level of the Angiogenic Response on the CAM | | | | | |
|---|---|---|---|---|---|---|
| | | Very | | | | Total > v. weak |
| | | weak | Weak | Moderately | Strongly | positive/No. tests |
| Sample | Negative | Positive | positive | positive | positive | (OVERALL LEVEL) |
| CONTROL | 18 | 7 | 1 | — | — | 1/26 (NEGATIVE) |
| DEAE (A-25, BIODEG) | — | — | — | 4 | 31 | 35/35 (STRONG) |
| 100 μg | | | | | | |
| DEAE (A-25, BIODEG) | — | — | 2 | 8 | 20 | 30/30 (STRONG) |
| 40 μg | | | | | | |
| DEAE (A-25, BIODEG) 8 μg | — | — | 1 | 7 | 2 | 10/10 (MODERATE) |
| DEAE (A-25) 100 μg | — | 1 | 10 | 9 | 2 | 21/22 (MODERATE) |
| DEAE (A-25) 40 μg | — | 2 | 14 | 10 | — | 24/26 (MODERATE) |
| DEAE (A-25) 8 μg | — | 3 | 7 | — | — | 7/10 (WEAK) |
| CM (C-25) 100 μg | 5 | 2 | 3 | 1 | 1 | 5/12 (NEG/V. WEAK) |
| CM (C-25) 40 μg | 10 | 1 | 7 | — | — | 7/18 (NEG/V. WEAK) |
| Sephadex (G-25) 100 μg | 6 | 5 | — | — | — | 0/11 (NEG/V. WEAK) |
| Sephadex (G-25) 40 μg | 8 | 3 | 5 | — | — | 5/16 (NEG/V. WEAK) |
| DEAE (A-50) 100 μg | 2 | 3 | 7 | 2 | 1 | 10/15 (WEAK) |
| DEAE (A-50) 40 μg | — | 7 | 5 | 3 | — | 8/15 (V. WEAK) |
| CM (C-50) 100 μg | — | 1 | 8 | 1 | — | 9/10 (WEAK) |
| CM (C-50) 40 μg | — | 4 | 9 | 1 | — | 10/14 (WEAK) |
| Sephadex (G-50) 100 μg | 1 | 2 | 3 | 1 | — | 4/7 (WEAK) |
| Sephadex (G-50) 40 μg | 2 | 1 | 3 | — | — | 3/6 (V. WEAK) |

It will be understood that various modifications may be made to the embodiments disclosed herein. For example the compositions described in the present methods can be blended with other biocompatible, bioabsorbable or non-bioabsorbable material. Therefore, the above description should not be construed as limiting, but merely as exemplifications of preferred embodiments. Those skilled in the art will envision other modifications within the scope and spirit of the claims appended hereto.

We claim:

1. A method of promoting angiogenesis comprising administering an effective angiogenesis promoting amount of a cross-linked polysaccharide having a positive charge to a desired locus, sufficient to promote angiogenesis in the desired locus.

2. A method of promoting angiogenesis according to claim 1 wherein the cross-linked polysaccharide is biodegradable.

3. A method of promoting angiogenesis according to claim 1 wherein the cross-linked polysaccharide is a bead.

4. A method of promoting angiogenesis according to claim 1 wherein the cross-linked polysaccharide is cross-linked dextran.

5. A method of promoting angiogenesis according to claim 1 wherein the positive charge on the cross-linked polysaccharide is provided by diethylaminoethyl groups.

6. A method of promoting angiogenesis according to claim 5 wherein the cross-linked polysaccharide is cross-linked dextran.

7. A method of promoting angiogenesis according to claim 6 wherein the cross-linked positively charged dextran is biodegradable.

8. A method of promoting angiogenesis according to claim 7 wherein the biodegradable cross-linked positively charged dextran is oxidized.

9. A method of promoting angiogenesis according to claim 1 wherein the cross-linked polysaccharide is contained in a composition having a pharmaceutically acceptable carrier.

10. A method of promoting angiogenesis according to claim 1 wherein the cross-linked polysaccharide is administered topically.

11. A method of promoting angiogenesis according to claim 9 wherein the composition is selected from the group consisting of powder, gel, ointment, paste, fluid and lotion.

12. A method of promoting angiogenesis according to claim 1 wherein the polysaccharide further comprises a medico-surgically useful agent.
